(19) United States
(12) Patent Application Publication
(10) Pub. No.: US 2024/0346647 A1
(43) Pub. Date: Oct. 17, 2024

(54) MEASURING HEAD, NECK, AND BRAIN FUNCTION AND DIAGNOSING MEMORY IMPAIRMENT

(71) Applicant: Stephanie Littell, Lexington, MA (US)

(72) Inventor: Stephanie Littell, Lexington, MA (US)

(21) Appl. No.: 18/631,855

(22) Filed: Apr. 10, 2024

Related U.S. Application Data
(60) Provisional application No. 63/458,537, filed on Apr. 11, 2023.

(51) Int. Cl. G06T 7/00; G06T 7/11

(52) U.S. Cl. CPC G06T 7/0012; G06T 7/11; G06T 2207/10081; G06T 2207/20084; G06T 2207/30016

(57) ABSTRACT

A computer based method and system for assessing brain frequencies in an individual includes using a near infrared spectroscopic device on an individual at an anatomical region to be studied. The method also includes determining, with the device at the anatomical region, a first frequency measurement of at least one molecule at a first time. The method further includes determining a second frequency measurement of the at least one molecule at a second time, and comparing the first frequency measurement to the second frequency measurement to generate a comparison. The method further includes identifying principal uncorrelated dimensions of the comparison to yield a covariance matrix, utilizing Bayes or other classification on the covariance matrix to yield a resulting conditional class probability, and thresholding the class probability to determine a classification for the individual's neural processing, the classification indicating a health status of the neural processing.

### RELATED APPLICATION

This application claims the benefit of U.S. Provisional Application No. 63/458,537, filed on Apr. 11, 2023. The entire teachings of the above application are incorporated herein by reference.

### BACKGROUND

Maintaining a healthy brain is critical to overall health and well-being. Early detection of disease or other threats to brain health permits individuals to seek treatment sooner and, ideally, provides individuals with a greater chance of reversal or prevention of further declines in brain health.

Memory impairment and memory loss can be a result of multiple etiologies. Commonly, Alzheimer's disease is a cause of memory loss. However, short-term or long-term memory loss may be caused by a number of other diseases and syndromes, such as medications, drugs, trauma, infections, dementia, depression, age-related diseases, and stroke. Progression of memory loss or memory impairment can be unsettling and debilitating. It is emotionally difficult for a person having memory loss or impairment, and such memory loss interferes with daily life. Memory loss or impairment ranges from the occasional forgetfulness (for example, where car keys were placed, whether medications were taken), to lack of recognition of friends and family members.

Memory impairment and memory loss also affects spouses, family members, relatives and friends who witness first-hand mental and cognitive decline.

Typically, Alzheimer's disease is clinically diagnosed based on family history and behavioral observations in a patient, for example assessed through neuropsychological tests. Certain other cerebral pathologies can be ruled out via MRI or CT imaging technologies. Definitive biological diagnoses of Alzheimer's disease are currently possible only upon autopsy and analysis of brain tissue, however, certain biomarkers for Alzheimer's disease allow for a probable diagnosis. Amyloid protein plaques, for example, are a biomarker of Alzheimer's disease and are detectable in cerebral spinal fluid.

An adequate supply of oxygen and nutrients is critical for normal brain function. Blood carrying oxygenated hemoglobin and nutrients is delivered to the brain through the vascular system, whereby pulsations of blood travel from the heart, through the carotid and vertebral arteries, and ultimately through capillaries where the exchange of water, oxygen, carbon dioxide and other nutrients occurs between the blood and nearby tissues. Decreased blood flow to a region of the brain may result in impairment of that brain region's function. The interruption of blood flow to a region of the brain of an individual may indicate that the individual has suffered a stroke, a condition for which immediate medical attention is required, even in the absence of outward physical symptoms.

Frequently, medical attention for the diagnosis and treatment of illnesses related to brain function is not sought until after the manifestation of physical symptoms, such as memory loss, impairment of speech, loss of consciousness, numbness, and paralysis. In the absence of timely medical attention, lasting disabilities or death may occur. The ability to visualize the vasculature and circulatory function of the brain is not accessible to the average individual, except through imaging studies conducted by medical professionals, and, individuals frequently delay seeking medical attention until after the onset of physical symptoms.

Therefore, there is a need for improved methods to monitor brain function, health, and memory, and particularly for methods which are accessible to the average individual. Additionally, there is a need to detect decreased or interrupted blood flow before the onset of physical symptoms. There is also a need for improved methods to predict and diagnose a patient's level of memory impairment and cognitive ability, and track a patient's memory loss or impairment over time.

### SUMMARY

The present invention relates to methods of monitoring and visualizing the vasculature system and circulatory function of the brain, as well as other characteristics, enabling individuals to monitor their own brain health. The present invention also relates to methods of alerting individuals without significant medical knowledge of potentially adverse changes in their circulatory function in an easily comprehensible manner, prompting them to seek early medical attention, if needed. The present invention further relates to methods of predicting and diagnosing memory impairment in an individual and to determining and monitoring the ability of an individual to form new memories. In addition, the present invention relates to computer systems and devices that aid in the monitoring of brain function, health, and memory.

Accordingly, in an embodiment, the invention is directed to a method for assessing brain frequencies in an individual. The method includes using, e.g. positioning, a near infrared spectroscopic device on an individual at an anatomical region to be studied. The method also includes determining, with the device at the anatomical region, a first frequency measurement of at least one molecule at a first time. The method further includes determining a second frequency measurement of the at least one molecule at a second time, and comparing the first frequency measurement to the second frequency measurement to generate a comparison. The method further includes identifying principal uncorrelated dimensions of the comparison to yield a covariance matrix, utilizing Bayes or other classification on the covariance matrix to yield a resulting conditional class probability, and thresholding the class probability to determine a classification for the individual's neural processing, the classification indicating a health status of the neural processing.

Another embodiment of the invention is directed to a computer system to assess brain frequencies in an individual. The computer system includes a measuring module configured to determine a first frequency measurement of at least one molecule at a first time and configured to determine a second frequency measurement of the at least one molecule at a second time. A comparison module is configured to compare the first frequency measurement to the second frequency measurement of the at least one molecule. The computer system further includes an identification module coupled to the comparison module and configured to identify principal uncorrelated dimensions of the comparison to yield a covariance matrix. A probability module is coupled to the identification module and configured to utilize Bayes or other classification on the covariance matrix to yield a resulting conditional class probability. The probability module is configured to threshold the class probability to determine a classification for the individual's neural processing, the classification indicating a health status of the neural processing.

The first time, at which the first measurement is determined, can be a first moment or a first time interval, which can be less than 5 seconds, and the second time, at which the second measurement is determined, can be a second moment or a second time interval, which can be less than 5 seconds.

The method can include converting the first and second frequency measurements to time-based signal samples or to a sequence flowcharting sample or both. The computer system can include a conversion module responsive to the measuring module and configured to (i) convert the first and second frequency measurements to time-based signal samples, (ii) convert the first and second frequency measurements to a sequence flowcharting sample, or both (i) and (ii).

The method can include, using time-based observations, identifying a maximum or minimum frequency from at least one of the first and second frequencies. The identification module of the computer system can be configured to, using time-based observations, identify a maximum or minimum frequency from at least one of the first and second frequencies.

The method can include comparing an average of the first and second frequency measurements against a threshold value. In the computer system, at least one of the comparison module and the probability module can be configured to compare an average of the first and second frequency measurements against a threshold value.

The first frequency measurement can include a series of first frequency measurements and the second frequency measurement can include a series of second frequency measurements. Comparing the first frequency measurement to the second frequency measurement can include comparing the series of first frequency measurements to the series of second frequency measurements to generate the comparison as a comparison matrix. In certain embodiments of the computer system, the comparison module can be configured to compare the series of first frequency measurements to the series of second frequency measurements to generate the comparison as a comparison matrix.

The method can include establishing a baseline from one or more signals of the at least one molecule at the first time. In certain embodiments, the computer system further includes a baseline module responsive to the measuring module configured to establish a baseline from one or more signals of the at least one molecule at the first time. The baseline can represent a brain frequency pattern profile of the individual. The baseline may also represent a memory map, e.g. a local memory map, of the individual.

The method can include normalizing the brain frequency pattern profile based on the individual's age, gender, or other characteristic. In certain embodiments, the computer system includes a normalization module configured to normalize the brain frequency pattern profile based on at least one of age and gender of the individual. The measuring module can include a baseline submodule and/or a normalization submodule.

In embodiments of the method and computer system of the present invention, the measured molecule comprises a nucleic acid, an amino acid, a sugar, a protein, a fatty acid, a nucleoside, a nucleotide, or combinations thereof. The protein can be hemoglobin; the amino acid can be any one of glutamate and gamma-aminobutyric acid (GABA).

The protein can be hemoglobin; the amino acid can be any one of glutamate and gamma-aminobutyric acid (GABA).

In embodiments of the method and computer system, the at least one molecule can include an amino acid, the amino acid being tyrosine or phenylalanine. In certain embodiments, the at least one molecule includes an amino acid, the amino acid being a chemically-derivatized amino acid relating to dopamine. For example, the chemically-derivatized amino acid can be L-dihydroxyphenylalanine (L-DOPA) or dopamine.

In other embodiments, the method includes measuring cerebral blood pressure, cerebral blood flow, cerebrospinal fluid pressure, cerebrospinal fluid flow, intracranial pressure, or combinations thereof. In certain embodiments, the computer system includes or the measuring module includes a second measuring module configured to measure cerebral blood pressure, cerebral blood flow, cerebrospinal fluid flow, intracranial pressure, or combinations thereof.

In certain embodiments, the anatomical region includes a forehead of the individual. In other embodiments, the region comprises a frontal, parietal, limbic, occipital, or temporal lobe of the individual.

In certain embodiments, the computer system includes a device module configured to connect one or more near infrared spectroscopic devices. The device module can be operatively part of the measuring module.

In embodiments of the method and computer system of the present invention, the near infrared spectroscopic device is a portable device, such as a cell phone, tablet, phablet, laptop computer, wearable aid, for example, a wristwatch, wrist cuffs, or footwear. The device can also be a stand-alone device or at least one mountable sensor that can be placed onto a body, or embedded into clothing, bedding, or other devices. In certain embodiments, the near infrared spectroscopic device includes a built-in camera and software for measuring the one or more ions, one or more molecules, or combinations thereof.

In another embodiment, the invention is directed to a computer-implemented method of assessing brain health in an individual. The method includes using a camera of a mobile device to capture a sequence of images at an anatomical region of an individual, utilizing a digital processor associated with the mobile device to process the captured images to obtain at least one of frequency, estimated concentration, and conversion rate information relating to an analyte of the individual, and to analyze the at least one of frequency, estimated concentration, and conversion rate information to obtain functional features relating to the individual's brain. The method further includes rendering a graphical representation of the functional features of the individual's brain on a screen of the mobile device.

In another embodiment, the invention is directed to a mobile device for measuring and displaying brain health in an individual. The mobile device includes a camera configured to capture a sequence of images of an anatomical region of an individual, a digital processor, and a screen configured to render a graphical representation of functional features of the individual's brain. The digital processor can be configured to process the images to obtain at least one of frequency, estimated concentration, and conversion rate information relating to an analyte of the individual, and to analyze the at least one of frequency, estimated concentration, and conversion rate information to obtain functional features of the individual's brain.

In embodiments of the method and device of the present invention, the invention is directed to acquiring images in the RGB (red-green-blue) visible light spectrum to capture images of the vasculature of the face, head and neck. In another embodiment, the invention is directed to acquiring images in the near infrared spectrum to capture images of vasculature and neural functioning beneath the skull. Methods of obtaining underlying vasculature functioning are known in the art. For example, Mehagnoul-Schipper et al, 2002 developed and applied near-infrared spectroscopy for evaluating brain task-related oxygenation changes. The invention is also directed towards deducing functional features of the vasculature including a frequency of blood pulsations through a vessel, a volumetric change of a vessel, a concentration of oxygenated hemoglobin, a concentration of deoxygenated hemoglobin, rate of oxygen dissociation, and rate of nutrient diffusion across a blood brain barrier. Additionally, at least one of frequency, estimated concentration, and conversion rate information relating to an analyte of the individual could be obtained. Examples of the analyte include, but are not limited to, phenylalanine, tyrosine and dopamine. The mobile device of the invention may be a wearable aid, cell phone, tablet, laptop computer, or mountable sensor(s), for non-limiting example. Other mobile or portable devices are suitable. The functional features can be computed for each of a bilateral subsection of the individual's anatomy and the functional features of each of the subsections can be compared to assess brain health or a circulatory deficiency of the individual. Functional features can be compared to functional features of a group or population to assess brain health of the individual. A cyclic vessel wall displacement, a vessel volume, cyclic pressure, an angular velocity and a corresponding wave energy can be estimated. The individual can be alerted to brain health, a circulatory deficiency, or combinations thereof.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic view of embodiments measuring a head and/or face area of an individual.

FIG. 2 is a flow diagram of an example embodiment of the present invention.

FIG. 3 is a flow diagram of an example embodiment of the present invention to calibrate analyte transient to oxygenation transient.

FIG. 4 is a flow diagram of an example embodiment to render graphical representations of functional features on screen.

FIGS. 5A-5C are schematic illustrations of a mobile device including an example user interface for visualizing brain internal waves and assessment.

FIG. 6 is a schematic illustration of a mobile device including an example user interface for further visualizing brain assessment and introducing an analogy for decomposing frequencies.

FIGS. 7A-7B are schematic illustrations of a mobile device including an example user interface for further visualizing the analogy and select brain frequency properties.

FIG. 8 is a schematic view of an example computer network embodying the present invention.

FIG. 9 is a block diagram of a computer node in the network of FIG. 8.

### DETAILED DESCRIPTION

A description of example embodiments follows.

The generation of an action potential is required for the formation of a memory in humans (mammals generally). Therefore, in order to assess the ability of an individual to form a memory, or alternately, in order to assess the likelihood for memory impairment in an individual, the embodiments of the present invention provide methods and apparatus for assessing the probability of a neuron to generate an action potential.

Example methods and systems for predicting a likelihood of memory impairment are described in U.S. Pat. No. 10,791,982, entitled “Methods of Measuring Head, Neck, and Brain Function and Predicting and Diagnosing Memory Impairment,” which are also described in a corresponding PCT Application, International Application No. PCT/US2015/028826, which published as WO2015/168579, the entire teachings of which are incorporated herein by reference.

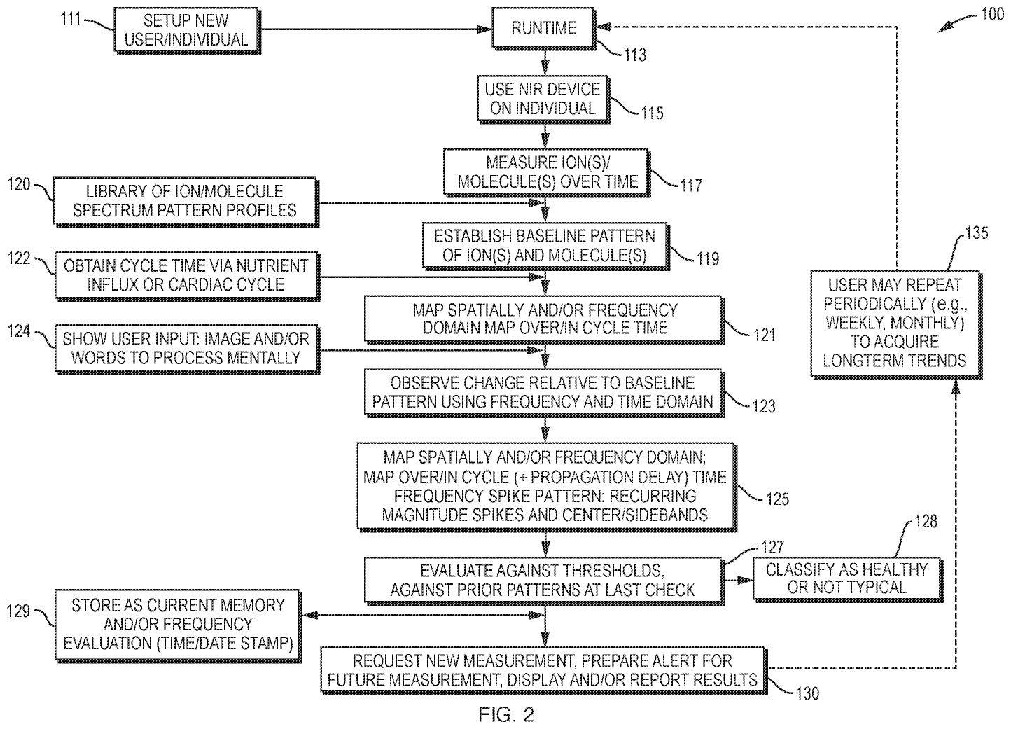

Measurements of analytes as obtained from near-infrared spectroscopy, such as oxyhemoglobin and deoxyhemoglobin, are known in the art. For example, as described in Strangman et al. 2002, spectroscopic information of sampled tissues can provide for the quantification of concentrations of various hemoglobin species, including oxyhemoglobin, deoxyhemoglobin, and total hemoglobin. As described in Strangman et al. 2002 and shown in FIG. 2 therein, the relatively low absorption factors of the primary light absorbers in biological tissue between roughly 650 nm and 950 nm provide for an “optical window” in tissue, through which changes in concentrations of oxyhemoglobin and deoxyhemoglobin can be detected. The absorption spectra of oxyhemoglobin and deoxyhemoglobin are known and, for example, are shown in FIG. 2 of Strangman et al. 2002. Since it is generally known that total hemoglobin concentration is a sum of the concentrations of oxyhemoglobin and deoxyhemoglobin, hemoglobin (Hb) concentration can be measured based on near infrared spectroscopy.

Methods of determining an oxygen concentration (PaO₂) and an oxygen saturation (O₂ sat) based on known concentrations of various hemoglobin species are also known and can similarly be obtained by near infrared spectroscopy. For example, as described in Saddawi-Konefka and Bryner, oxygen content (e.g., oxygen concentration, oxygen saturation) in blood can be determined based on hemoglobin saturation (i.e., a fraction of oxyhemoglobin to total hemoglobin).

As further examples, the spectra of carbon dioxide (CO₂) and carbonic acid (H₂CO₃) are known in the art, as provided, for example, by White et al., 2012 and Huber et al. 2012, and are known to be measurable within the infrared range. The spectra of hydronium ions (H₃O⁺) are known in the art and are known to be measurable within the infrared range, as provided, for example, by Biermann and Gilmour 1959. It is also known that hydrogen ions (H⁺) bind to water in aqueous solutions, such as blood, thereby being found as the hydronium ion in aqueous solutions.

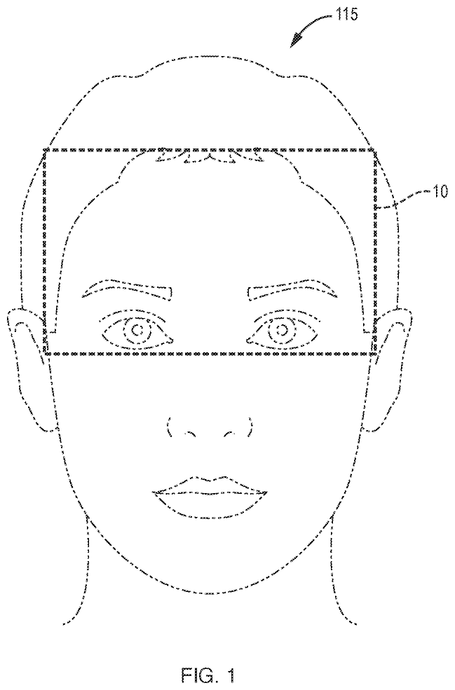

Embodiments of the present invention are directed to a method, systems and devices for assessing brain frequencies in an individual. An embodiment 100 illustrated in FIGS. 1 and 2 includes using a near infrared spectroscopic device on an individual (step 115 of FIGS. 1 and 2) at an anatomical region 10 to be studied. The computer-based system 100 is initialized for the individual at 111 and started at 113 of FIG. 2. The system 100 at step or module 117 determines, with the device at the anatomical region 10, a first measurement of one or more ions, one or more molecules, or combinations thereof at a first time. In an embodiment, the system determines a first frequency measurement of at least one molecule at a first time. Measuring module or step 117 further determines a second measurement of the one or more ions, one or more molecules, or combinations thereof at a second time. In an embodiment, the system determines a second frequency measurement of the at least one molecule at a second time. A comparison module or step 123 compares the first measurement to the second measurement of the one or more ions, one or more molecules or combinations thereof. In a particular embodiment, the comparison module or step 123 compares the first and second frequency measurements to generate a comparison. The system/method 100 (through a series of steps 119-125) assesses and eventually determines 127 (identification module), based on the comparison at 123, a probability for one or more neurons to generate an action potential (probability module). The identification module 127 can be configured to, using time-based observations, identify a maximum or minimum frequency from at least one of the first and second frequencies. Ranges of values for healthy brains can be established and individual might be classified as healthy or not normal (step 128). The generation of an action potential increases the probability of forming a new memory, and thereby is a predictor of or forms a prediction of the likelihood for memory impairment in the individual. Step 129 stores an indication of the prediction (evaluation results) 127 as a local-to-regional memory map. The system or device can include a conversion module responsive to the measuring module and configured to (i) convert the first and second frequency measurements to time-based signal samples, (ii) convert the first and second frequency measurements to a sequence flowcharting sample, or both (i) and (ii).

“Memory impairment” as used herein, means any problem with an individual's memory (e.g., a decline in an individual's ability to form new memories or ability to recall formed memories). Memory impairment can also refer to a deficit that is beyond the scope of what would be anticipated or predicted in the normal course of aging. Memory impairment can be assessed based on clinical observation, neuroimaging, neuropsychological testing, and so forth. In certain embodiments, an individual's memory impairment is compared against, or fit into a spectrum of memory impairment observed in other individuals, as stored in a library 120 for example. Such multi-dimensional fitting could be stored in the final memory evaluation 129 and used for further external processing. Such a comparison, or such a spectrum, can include an analysis of age, gender, education level, profession, physical fitness, past medical history, or other characteristics of an individual. In other embodiments, memory impairment is compared to or assessed against (in module or step 127) another measurement or prediction of memory impairment of the same individual, taken at an earlier time point, for example, five years prior.

In certain embodiments, the time at which module or step 117 takes a measurement of ions, molecules, or a combination thereof is a moment in time. In certain other embodiments, the time is an interval of time. The interval of time can be selected based on nutrient influx (e.g., Hb-O), or, alternatively, by arterial pulse cyclicality as indicated or otherwise provided at 122.

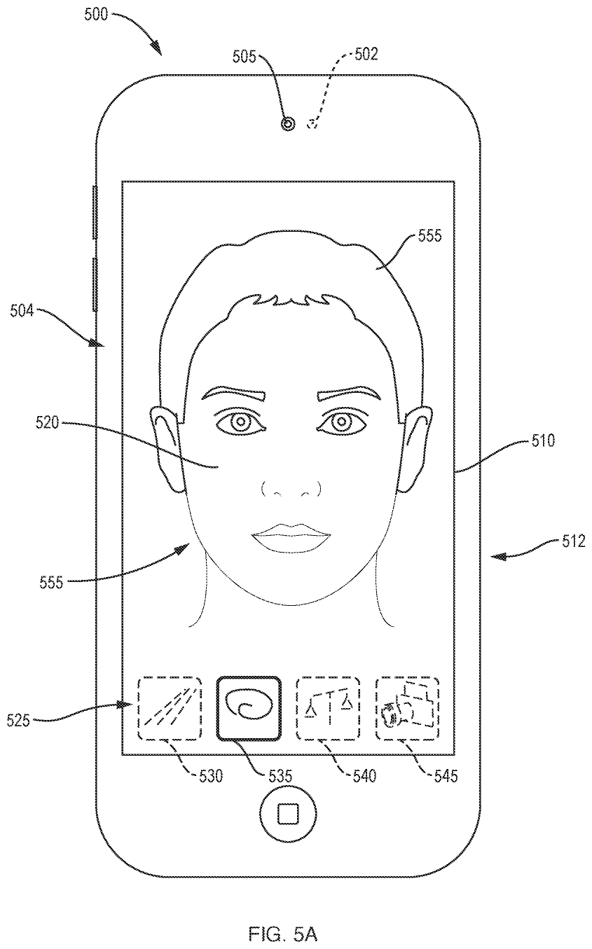

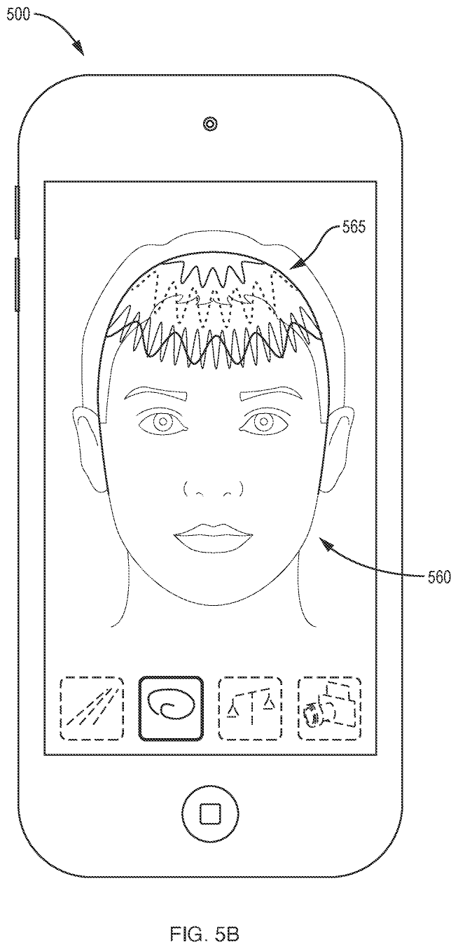

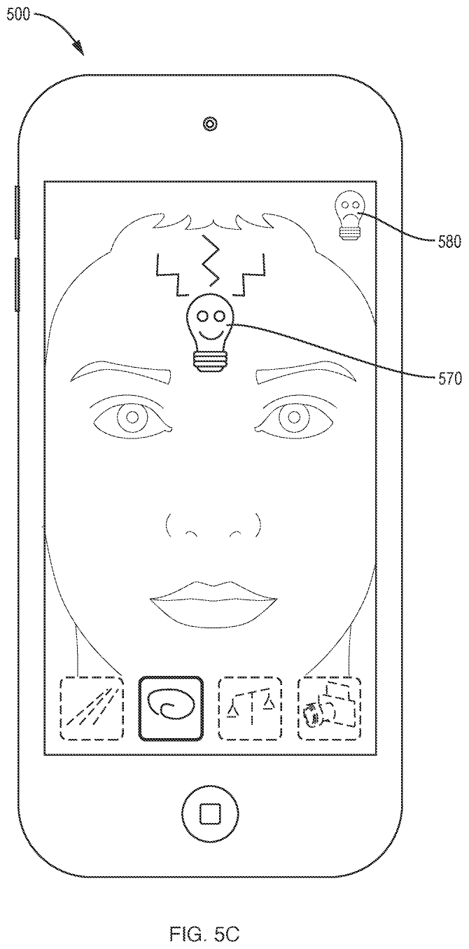

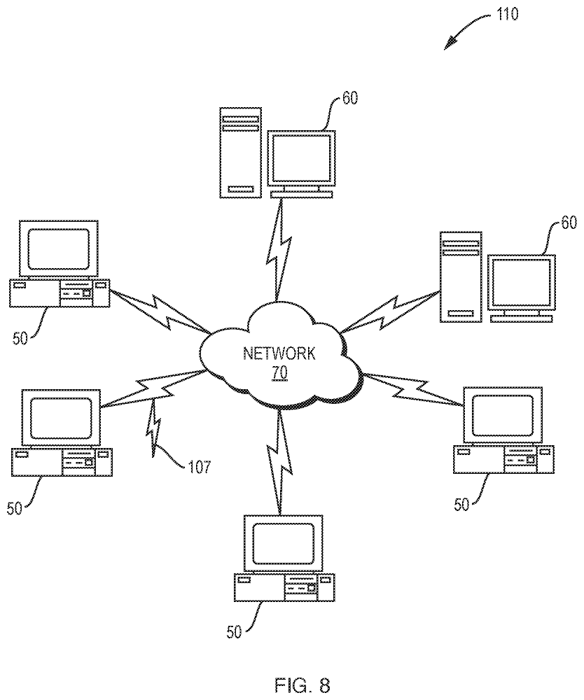

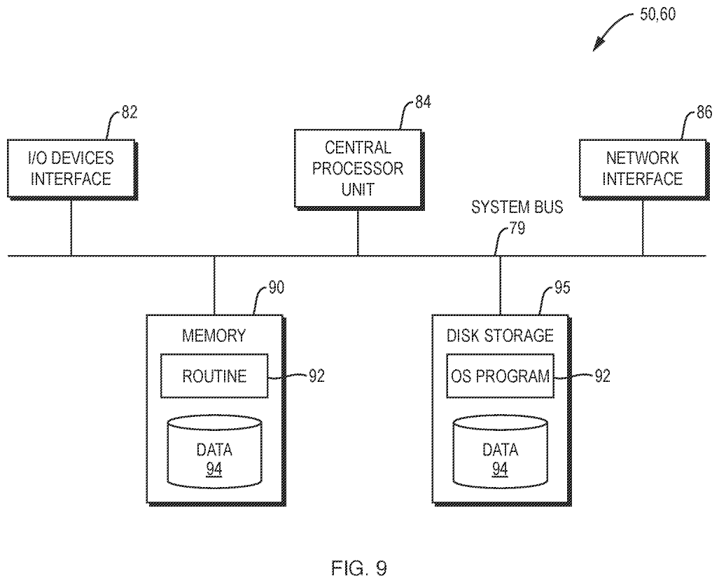

An infrared spectroscopic device of module/step 115 measures energy in the near-infrared (NIR) region of the electromagnetic spectrum (energy having a wavelength from about 650 nm to about 1400 nm). In certain embodiments, the device is portable. A spectroscopic device of 115 can be connected to or be part of an electronic processing device (e.g., cell phone, tablet, laptop computer, portable digital processor device, or handheld computer) 50 of FIGS. 8 and 9, such as the mobile device 500 of FIGS. 5A-5C, 6 and 7A-7B. In certain other embodiments the spectroscopic device at 115 further comprises a screen display or monitor. In other embodiments, the spectroscopic device 115, 50 comprises a module for emailing or uploading data to a server 60 (FIG. 8), a computer connected to the Internet 70 (FIG. 8), or a printer (or similar I/O devices). In other embodiments, the spectroscopic device 50, 115 comprises a camera and programmed processor for measuring one or more ions, one or more molecules, or combinations thereof.

The anatomical region 10 that is studied in the methods and systems of the present invention can lie anywhere along the surface of the head as schematically shown in FIG. 1. In certain embodiments, it is most efficient to begin with the prefrontal cortex, which is often furthest from the cardiovascular supplying arteries. The prefrontal cortex also contains the highest-level integration of neuronal information, manifested as executive functions. Other cerebral cortex regions may also be assessed.

In certain embodiments, the anatomical region 10 that is studied is a forehead. Area 10 outlines the approximate region to measure an ion concentration and/or flux. In certain other embodiments, the region comprises a frontal, parietal, occipital, limbic, or temporal lobe of the individual. The spectroscopic device may also be used at more than one site on the individual. For example, depending upon the results at one site, the user may reposition the device to another cerebral region (generally 10), or another head or brain region (generally 10) altogether.

As previously described in reference to FIG. 2, the method and system 100 include the determination of a measurement 117 of one or more ions, molecules, or combinations thereof. Such a measurement 117 is taken at two or more times (e.g., 2, 3, 4, 5, 6, 7, 8, 9, 10, 15, 20, 25, 30, 50, 100, etc.), the measurements are compared 123, and this comparison enables the determination 127 of a probability for one or more neurons to generate an action potential. The ability of a neuron to generate an action potential increases the probability of forming a new memory.

In certain embodiments, the measured molecule is a nucleic acid, an amino acid, a sugar, a protein, a fatty acid, a nucleoside, a nucleotide, or combinations thereof.

In certain embodiments, the nucleic acid is a polymeric macromolecule or biological molecule. Nucleic acids include deoxyribonucleic acid, (DNA), ribonucleic acid (RNA), or artificial analogs of nucleic acids.

In certain embodiments, the amino acid is a naturally occurring amino acid or an artificial amino acid. Naturally occurring amino acids include essential amino acids histidine, isoleucine, leucine, lysine, methionine, phenylalanine, threonine, tryptophan and valine. Non-essential amino acids include alanine, arginine, asparagine, aspartic acid, cysteine, glutamic acid (or glutamate, the deprotonated form of glutamic acid), glutamine, glycine, ornithine, proline, selenocysteine, serine, and tyrosine. Amino acids can also include synthetic amino acids, or chemically derivatized amino acids, such as L-dihydroxyphenylalanine (L-DOPA). In certain embodiments, the amino acid is gamma-aminobutyric acid (GABA).

In certain embodiments, two or more amino acids are linked, forming a polypeptide that is measured. In some embodiments, the measured polypeptide is a protein. In certain embodiments, the protein that is measured is hemoglobin.

Sugars measured by embodiments include monosaccharides, disaccharides, and polysaccharides. Sugars can exist in linear chain or cyclic configurations, and include, but are not limited to glucose, sucrose, fructose, maltose, galactose, and lactose.

In certain embodiments, the fatty acid that is measured is a carboxylic acid having a long saturated or unsaturated aliphatic chain. Fatty acids include, but are not limited to, linoleic acid, alpha-linolenic acid, eicosapentaenoic acid, docosahexaenoic acid, oleic acid, elaidic acid, vaccenic acid, linoclaidic acid, arachidonic acid, erucic acid and so forth.

A nucleoside comprises a nucleobase (e.g. adenine, guanine, thymine, uracil, cytosine), bound to a 5-carbon sugar (e.g. a ribose or a deoxyribose in a pentose conformation), via a beta-glycosidic linkage. In example embodiments, the measured nucleoside is cytidine, uridine, adenosine, guanosine, thymidine or inosine, where the nucleoside contains either a ribose sugar component or a deoxyribose component.

In certain embodiments, a nucleotide comprises a nucleoside linked to one or more phosphate groups. In certain embodiments, the nucleotide that is measured comprises ATP, ADP, GTP, CTP and UTP, cGMP, CAMP, coenzyme A, FAD, FMN, NAD, or NADP+.

In certain embodiments, the measured ion is hydrogen (H⁺), sodium (Na⁺), potassium (K⁺), calcium (Ca²⁺), magnesium (Mg²⁺), chloride (Cl⁻), carbonate (CO₃⁻), bicarbonate (HCO₃⁻), or a phosphate (H₃PO₄, H₂PO₄₋, HPO₄²⁻, or PO₄³⁻).

The ability of one or more neurons to generate an action potential is affected by conditions and parameters, as laid out in detail below, an assessment of which occurs in module 123. These conditions and parameters include: A. the concentration and flux of ions, B. acidosis, C. the concentration and flux of certain molecules, D. hypoxia, and E. cardiovascular parameters.

Returning to FIG. 2, the methods and apparatus 100 of the present invention also provide for the assessment at module 127 of hypoxia, acidosis, ion flux, or combinations thereof. Such an assessment is based on using the near infrared spectroscopic device 115 to determine one or more measurements 117 (including a baseline pattern 119) of one or more molecules, ions, or combinations thereof. Specifically, hypoxia is determined to be present based upon measured oxygen concentration (PaO₂), oxygen saturation (O₂ sat), arterial oxygen content (CaO₂), hemoglobin (Hb) concentration, or combinations thereof. A measured concentration of carbon dioxide (CO₂) can also be useful in the detection of chronic hypoxia because an elevated concentration of CO₂ would eventually not spur an increase in respiratory oxygen through to a sensed region of the brain (through the vasculature and the blood brain barrier to CSF at the brain stem). Oxygen in a healthy individual would increase quickly (within approximately 1-3 seconds), in response to an elevated concentration of CO₂, via increased respiratory action. Although it is possible that an elevated level of CO₂ could initially manifest at the exact time of testing in a healthy individual and before a corresponding rise in O₂, a detection of elevated CO₂ is more likely to indicate ineffective O₂ delivery and/or chronic hypoxia. Acidosis is determined to be present based upon measured concentration of hydrogen (H⁺), bicarbonate (HCO₃⁻), carbonic acid (H₂CO₃), CO₂, phosphates (PO₄³⁻, HPO₄²⁻, H₂PO₄, H₃PO₄), or combinations thereof.

As discussed above, persons with memory impairment suffer from a physiological inability or lowered ability to form new memories. A neurological memory is a process in which patterned input 124 is encoded, stored, and then later retrieved by an individual. Memories are formed and stored when neurons generate an action potential, for example a synaptic potential, or fire sufficiently above the action potential threshold, traversing from one presynaptic neuron to the next. Neurons manage electrochemical gradients such that reasonably small amounts of various ions can influence the cross-membrane potential. In an example embodiment, an appropriate action potential generates, and is followed by, a downstream recording event, or memory production.

Certain areas of the brain have been identified as being involved in memory, function, and storage, including the cerebral cortex, cerebellum, hippocampus, basal ganglia, amygdala, the striatum, and the mammillary bodies. Certain areas of the brain are thought to be involved in specific types of memory. For example, the hippocampus is believed to be involved in spatial learning and declarative learning, while the amygdala is thought to be involved in emotional memory.

There are several types of memories that are implemented by the brain in distinct ways. Working memory is the ability of the brain to maintain a temporary representation of information about a task that an animal or individual is currently engaged in. This sort of dynamic memory is thought to be mediated by the formation of cell assemblies or groups of activated neurons that maintain their activity by constantly stimulating one another.

Episodic memory is the ability to remember the details of specific events. This sort of memory can last for a lifetime. There is evidence that implicates the hippocampus in playing a crucial role in forming episodic memory. For instance, people with severe damage to the hippocampus sometimes have amnesia, the inability to form new long-lasting episodic memories.

Semantic memory is the ability to learn facts and relationships. This memory is probably stored largely in the cerebral cortex, mediated by changes in connections between cells that represent specific types of information.

There are many diseases, conditions, and other reasons that may cause memory impairment or loss, e.g., amnesia. Commonly, memory impairment or loss is associated with Alzheimer's disease. Other causes of memory loss or impairment may be from head trauma, drugs, alcohol, infections (e.g., encephalitis, HIV, Lyme disease), cardiovascular disorders (e.g., stroke, transient ischemic attack), psychological disorders (dementia, depression), neurological disorders (e.g., epilepsy, Parkinson's disease, Huntington's disease, multiple sclerosis), cancer (e.g., brain tumor), nutritional-deficiency (e.g., Vitamin B12), and aging.

For an individual suffering from memory loss, or the inability to form new memories, the study of certain chemicals in the brain can provide insight into the conditions underlying memory loss. As is detailed below, both Alzheimer's disease and conditions that cause an inability for neurons to form an action potential are, in certain embodiments, tracked or understood through analysis of particular ions and molecules.

In order for neurons to fire, cellular energy fueling on-demand must occur, from corresponding oxygen delivery via blood's hemoglobin. Different firing frequencies entail differing rates of oxygen uptake (as it's demand-driven for cellular ATP production efficiency).

The five categories of brain frequencies in order of highest frequency to lowest are: gamma, beta, alpha, theta and delta. Conventionally gamma is greater than 30 Hz, beta is 13-30 Hz, alpha is 8-12 Hz, theta is 4-8 Hz, and delta is less than 4 Hz. By for example tuning in to gamma versus beta frequency, brain processing could be observed to “switch” or modulate from one to the other. if performing certain mental tasks. Beta rhythms in a scientific experiment control pieces of information, such as clearing out from working memory. Gamma rhythms can hold pieces of information in working memory.

Frequencies modulate on a finer-grained level within each respective frequency band, as in FM radio. Frequency and phase modulation, rather than amplitude modulation (e.g., AM radio) carrier, is more robust against noise. Thus, pieces of information are encoded or retained by using specific frequencies within a brain frequency band. Sampling for the spectrum of frequency spikes becomes a means to observe brain processing for a mental task. As a task progresses through time, temporal shifts in frequency spikes occur. Then typical processing for mental tasks can be classified, as can atypical processing.

For an individual, frequency and temporal-based processing patterns for a given mental task or physical activity could shift over time. For example, due to increased blood flow (headache, stroke), decreased blood flow (stroke, memory loss), inconsistent neuronal firing (ADHD), arising disorder (schizophrenia, psychosis), developmentally (as decision making matures), or perturbation in planned motor movements, change from individual baseline may be noted.

For some individuals, the full breadth of carrier frequencies cannot be employed, perhaps due to disorder or disease. Higher carrier frequencies (as for lower) can impart (energy and) signaling through envelope and internal modulated frequencies.

Tasks can involve ‘change’ steps, and perhaps even stasis steps. The finer-grained task change steps that can take place, the more encoding for change must necessarily occur. Task steps that change less entail lower frequencies in time. Task steps that change more finely employ higher frequencies, in order to encode more nuanced change steps in nuanced modulations to a frequently-peaking carrier wave. Handwriting, weighting multivariate decision-making, or at the onset of new walking or cycling decision making, for example, involve many fine-grained steps to complete such task.

To encode a finer-grained task, a person typically needs to employ higher-frequency carrier waves. The higher-frequency carrier wave envelope is modulated by frequencies representing the task step change encoding necessary to perform the task. If higher-frequency carrier waves such as gamma are not fully or consistently employed for example in a finer-grained learning task, the person may not fully encode task aspects (ADHD, learning disorders, Parkinson's Disease). To detect this issue, the application can simply derive the maximum frequency spike values manifesting through time.

Further involving carrier frequency detection, as a person ages, their heart stroke output lessens and heart muscles stiffen among other factors, decreasing each heart pulse wavefront's amplitude, angular momentum and HbO concentration. This leads to lessening ability to achieve higher imparted energy delivery via transduction from circulatory system to brain. Neuronal firing frequencies can be generated as a function of cellular energy. This also leads, as in Alzheimer's, to lessening fine-grained modulation (as brain frequencies cannot consistently and independently gain increased energy, i.e. rhythmic base frequency, without sufficient energy delivery and transduction). To detect this issue, the application can simply derive the maximum frequency spike values manifesting through time.

As modulated frequencies are carried with the underlying carrier frequency (whether conventionally gamma, beta, alpha, theta and delta band range), such signal could be decomposed into its carrier and modulated components. Deconvolution via say Discrete Fourier Transform (as instrumentation noise involved) would need to iterate through possible signal range curves for underlying carrier wave(s), in order to yield a modulation signal curve of frequencies (and phases) representing the observed neural encoding. (Such iteration boosts with usage patterns.) This modulation frequency curve can also be converted to the time domain if desirable. Such discrete or continuous curve(s) can be compared to others for a similar mental or physical task, or to the same individual through elapsed time.

Such individualized frequency or time-based modulation curve(s) allow for comparisons by task, whether sensory, purely mental, physical actions or a combination as the brain is always involved. For a given mental or roughly equivalent task, modulation curves can be correlated to change steps performed. Curves can be allocated into bins of range values, perhaps after differencing from prior individuals' or grouped results to preserve outliers. Curve mean or average magnitude per bin can be estimated, yielding magnitude per change step boundary or middle. Results can be compared across one's prior sampling, individuals, or across groups.

Before displaying relatively detailed results, it is better to gradually introduce an individual to his brain's inner fluctuations. First perhaps a rotating dominant wave (e.g. beta or gamma) displays moving around a central point as during consciousness. Gradually other lesser carrier waves from other bands might be shadowed in background.

Rotating carrier waves enable energy-efficient modulation in order to carry information. If discontinuities, disorganization, or even cessation of the (dominant) carrier wave occurs, then memory and/or mental processing can be impaired or disrupted. Thus, application observation is relevant to detecting processing issues.

Since multiple waves may be traveling, at a given point observation occurs and can be processed as a 2D Fourier transform, yielding spatial frequencies in image integration over I(x, y). The spatial frequencies can then be evaluated together to yield plane wave propagation directionality. The time evolution of directionality can then be evaluated (presuming typical skull bounds), yielding directionality (or directionalities) over time. If spatial directionality is not steady or typical, then application observation is relevant to detecting processing issues.

Alternatively, a set of image points could be processed as a 3D transform, via image projection to transform slice. Spatial directionality of multiple waves could be assessed, and issues flagged.

Waves indicate the existence of evolving electric fields arising collectively from cells, and as such evince signal energy delivered at a rate of signal power. From the frequency domain, carrier wave and modulated components' areas under the curve can be squared over the frequency range to yield energy. However, if the carrier wave component is cyclical its energy is infinite and so not as meaningful as computing power by virtue of Parseval's theorem in averaging such square(s) over the time interval sampled. Such application display becomes meaningful in aging, or declining conditions, e.g. Parkinson's disease.

Relatedly, an individual may discover that he is not forming memories as he used to, or in classroom/work setting is unable to learn, i.e. form pertinent memories. Noting power rate (or energy) during such time period, and comparing to previous samples for this declining individual, can address the arising deficiency in necessary energy for memory formation. For the class/work setting presuming such deficiency is steady-state, comparing power rate (or energy) to other like individuals would be useful. In both scenarios, the deficiency can be quantified.

With such quantification, energy boosting delivery tools could be developed that would deliver timed energy bursts consonant with cyclic carrier waves. e.g. light, sound, electric or magnetic fields, maybe medicines

The application may also choose to display frequency components that modulate observed task change and hold steps that the individual experiences, or a selection thereof. The app may do so by cycling through frequency spikes as the individual processes the task, or by cycling through their equivalent signal representations in time domain, or both. Then again, such observations can be earmarked as typical, or atypical. Results may be packaged more attractively as marked to a hypothetical person, someone typically relevant by being similar to this individual, e.g. gender, age, similar processing tasks, similar processing results.

In the prefrontal cortex (PFC), executive control is maintained by activity patterns that represent goals. As goals shift, the PFC acts in ways to shift activity elsewhere in the brain. For example, the brain can shift attention away from repetitive mundane aspects observed to new ones. The modulating signals blend with the frequency of the sinusoidal carrier wave, where phase (or derivative of proportional angle with the modulating signals) shifts. These changes can evince in shift in (net) wave directionality perhaps, or in modulated frequencies and phase shifts that occur in real time that is marked to the premised goal change. With goal testing and use of both time and frequency modalities, the application could detect whether an individual is typical or atypical (as in autism) in processing a goal change. Further, as excitatory dopamine release can override release of inhibitory neurotransmitters, such dopamine release involvement in goal or task shifting can be seen via near infrared spectra windowing.

Relatedly, to appeal more broadly, to those who are less concerned about their processing performance, a ‘competition’ or virtual ‘race’ can take place.

Using the above for Hb, and repeating a Hb [time2]−Hb [time1] iteratively over a sampling interval (while a mental task is being performed), yields a time-based signal that represents the change in hemoglobin uptake in a region. Hb uptake correlates to neuronal processing demands in that region. The time signal can then be converted via Fourier transform or Fast Fourier transform (FFT) into a frequency-based signal, to show which frequencies are utilized, and how they change.

Aperiodic signals in time can be represented by their Fourier counterpart in frequency spectrum treated over an infinite range. Brain signals are likely aperiodic in encoding mental processing or decoding such, as mental tasks or most other lived experiences transpire aperiodically.

The Fourier transform of the (observed and preprocessed) signal can be obtained. Such spectrum transform then represents the carrier frequency signal convolved with the content encoding/decoding.

In the spectrum transform curve, the carrier frequencies can be sought by selecting the largest magnitude frequency spikes. If such spikes present with lessening frequency values in the transform curve nearby, then those lesser values can be labeled as sidebands to the center frequency, and the amount of maximum frequency deviation from the peak frequency derived. (This is necessary because as the carrier frequency increases or decreases, sideband properties need to grow or shrink accordingly to function well.) A modulation index reflects this general need as d=Δf/fm, where the latter is the maximum frequency in this center+sidebands channel. Identifying the modulation bandwidth per center frequency channel could be useful where roughly bandwidth=2 (d+1) fm, and then any issue with distance or overlap between them. (Modulation index useful for identifying whether narrowband where d<<1 and needs less bandwidth, versus wideband where d>>1 and needs bandwidth of approximately 2 Δf.)

As the carrier wave modulates together with the content encoding or decoding, the change of phase in the content signal or phase derivative, is proportional to the original content. A bandpass filter can re-center the modulated signal to baseband, then a polar discriminator can de-modulate complex values by multiplying the prior time sample's frequency conjugate by the current time sample's frequency. Then the angle is extracted, yielding the instantaneous frequency of the signal.

Another frequency modulation encoding method is quadrature encoding, which employs phase shifts of 90 degrees. In other words, a delay in onset relative to an initial wave, such as occurs with phasic neuronal bursts in the prefrontal cortex when learning or decision-making. This could be implemented via a radio mode button that would display similarly to sample decoded readout of instantaneous frequencies in time, with perhaps a “learning well’ alongside for number of instances per week etc.

In the spectrum transform curve, there may exist frequency spikes that do not belong to a center/sideband set, nor belong to a repeat cyclic train. Such “individual remainder” spike(s) may represent additional frequency signals that are blended in with the above, and might additionally be increasing or decaying over time. Perhaps patterns could be identified in these remainder spikes, such as individual impulses versus increasing or decreasing curves in frequency. Since the brain is presumably mostly causal, involving rational rather than complex numbers, presumed usually as a linear constant-coefficient system, and also stable, it could be inferred that the underlying region of convergence involves zero or greater real numbers. Thus, a frequency or set of frequencies and their magnitudes could be pattern matched in a time moment or interval. Iteration may need to occur, as frequencies may belong to a different set than initially tagged.

Upon successful tagging of all obtained frequency sets, inverse Fourier transforms back to time domain could result. Such time-based signal sample imputed from observation intervals could be made available onscreen.

In the frequency domain, any numerator and denominator polynomials (in continuous time) or exponentials (in discrete time) for a transfer function between input and output signals could thus be of the form 2 sin [w(N1+½)]/sin (w/2) for one term in the discrete Fourier domain (where w represents angular frequency). In this case conversion to time domain would then yield x[n]=1 for |n|<=N1, 0 for |n|>N1.

Any numerator zeroes of a tagged frequency set's imputed term would increase over observed time. Denominator poles, e.g. 1/(jw+1), would decrease. Assorted terms represent parallel or series functions that are additive or multiplicative linear transforms respectively. Using transform properties, a sequence processing flowchart e.g. feedback loop, integrator, adder, cascade etc., could then perhaps be constructed. For example, a flowchart could be drawn based on a tagged term 1/(jw+1): input into an adder that flows into a functional block (1/jw) then splits to yield output or a functional block (−1) back to the adder. This represents a sample bounded by time intervals seen, but displayed in sequence flowcharting it might aid in people's comprehension.

Perhaps a power estimate per channel band would help some people, e.g. elderly, those with processing problems.

A convolution in time domain transforms to a multiplication of Fourier series coefficients in frequency domain (or vice versa). Thus, to unpack encoding content from the carrier signal, their Fourier coefficients can be decomposed (i.e. divided) from the carrier coefficients. Then such content-only Fourier spectrum can be inverse-transformed back to time domain.

To do so, iterative guesses need to be performed with carrier frequency values (since individuals vary), using application contextual awareness of a mental or physical task being performed concurrently. For example, for high performance mental or physical actions, gamma range would be iterated through.

Iterating finds suitable frequencies that, via frequency selection and division operations, decompose possible time-responsive content curves.

In order to identify one or more sufficiently good time-responsive content curve, such candidate curve would contain arising signal values that correspond to mental task steps in time, eg. signal as a series of impulses, square waves, ramps, steps, or combination components, expressed over time axis where each arising component occurs at time [mental task step+t].

An individual's content curve for a set of mental or physical task steps, can then be compared against prior same or similar content curves for this individual, or for a group, or for a population. A comparison can be assimilated with other comparisons, to deduce an assessment or set of assessments regarding mental performance and possibly dysfunctions. For example, the wave patterns in the first year of life may reflect possible autism, if steep increases in delta band and slower increases in gamma power than typical children.

Another comparison against populations can be made if wave patterns demonstrate a delta-brush or perhaps a beta-brush, where higher frequencies are grafted onto slower underlying wave patterns. This can indicate encephalitis or brain inflammation, often affects younger people in various forms, e.g. meningitis, West Nile virus, or perhaps even with Covid for younger or older.

Mental task change steps that can be more fine-grained in time (such as decision-making or handwriting), involve time-shortened processing. The shorter in time that each step transpires, the more its internal representation needs to become closer to an electrical observed pulse than a longer rectangular pulse in time, as the next step will soon arise. As the signal expresses a time evolution involving each necessarily aperiodic impulse, the Fourier frequency domain will express increasing spectrum for each. Thus, such frequency spectrum can be observed for any deficits in internal range, e.g., ADHD.

Spontaneous bursting of select neurons seems to occur in the neocortex, according to neuroscience research in juvenile mice. These bursts arise without external stimulation (in isolated neocortical slices), effectively creating an impulse train. One such neuron subtypes is a pyramidal neuron subtype located in layers 2/3 and 5. (Another is an interneuron with ascending axon projection in layer 5) A periodic “unit” impulse train converts via Fourier to an impulse train in the frequency domain, where as the time period lengthens, the impulse frequency interval compresses. When such oscillations progress to synchronous in the impulse train, frequency and then phase coalesce, according to another paper. As the waves' phases progress to synchronize, the waves center their superpositions, and also reinforce in amplitude (rather than sometimes perhaps, cancelling each other out when phases are different). Encoding would become sharper, as would decoding (information retrieval) at an aligned single frequency. Sharpness is related to the quality (Q) of the obtained circuit, where the magnitude of the frequency response is large and narrow in frequency. This can be examined by viewing the frequency spectrum in a high-demand mental task, where a unique spectrum spike would be seen in a phase modulo cycle.

Synchronization as above, with zero phase characteristics would indicate no phase distortion of a signal convolved with that oscillation train. If an impulse train were to repeat in frequency analysis with the same Fourier magnitude say every 2pi*(some base frequency) (where angular frequency=2*pi*linear frequency), then that represents the cyclic oscillation train. Remaining Fourier curves' spike magnitudes can be divided by the (removed) cyclic impulse magnitude, yielding the decomposed content-only Fourier signal magnitude. Then such content-only Fourier spectrum can be inverse-transformed back to time domain.

Synchronization or phase coupling could represent one kind of transfer from sets of neurons (subsystem) to other sets of neurons, as no phase distortion exists. (Phase to amplitude coupling could represent another, power coupling between bands yet another.) Thus this phase-distortion free condition can be noted by the application when it occurs. Phase can be obtained perhaps most easily by using the FFT where output is a complex number including frequency magnitude and phase. Identifying repeating phase values in the coupling cycles, allows for earmarking the combined carrier and modulating signal included in the pattern. If the derivative of the phase angle varies proportionally with the modulating signal, then the [frequency magnitude, phase] data points can be assembled. Using the instantaneous frequency in time (as it changes from one value to another), then subtracting the corresponding carrier frequency coordinate yields the modulating signal times a constant that is frequency-dependent. dθ/dt=w_{c}+kf x(t), where θ represents phase angle, and x(t) represents modulating signal.

It might be useful to see where Fourier transform magnitude gain is greatest, i.e. at which frequency narrowbands. Perhaps this is where encoding could be strongest, such that it impresses upon the individual. Contrasting to others at such bands could impart understanding of one's significant weighting. Such narrowband frequency may need to be translated, i.e. explained to the individual if correlated to a subtask in scope.

Alongside Fourier magnitude when encoding, phase shift can denote edge demarcation or boundary of some sort. This would be useful to denote, relative to subtask performed, for example perhaps when a new sequence of encoding begins or ends.

For a narrowband frequency of note (e.g. greatest gain as above), estimating rise time in time domain or group delay in frequency domain and comparing to others at such bands could also be useful. Unwrapping could be useful in specific cases for plotting distortions, if could be correlated to actual subtasks.

Encoding at a change of narrowband frequency (e.g. new task) might entail a gradual dropoff in ‘old’ frequency and transition to new. Filtering characteristics might be useful to quantify, such as the Fourier magnitude drop-off by frequency, and imputing by reverse transform any step characteristics in the new passband (e.g. ringing, overshoot).

Overall insight could be provided via a Bode plot of magnitude and phase. It would be based upon sampling to date, therefore utilizing cumulative data acquired and refining or adjusting over time. Comparing to others or to a typical sample sorted by group could be useful for the layman.

On-screen displaying by narrowband frequency for this drilldown aspect, could present via a FM-type dial. Instead of numbers, task icons or types could show on tuning dial or slider.

### Example Procedural Steps

Camera application on personal device is launched and set to video mode. Frame rate is set to max, e.g., HD1080 at 60 fps.

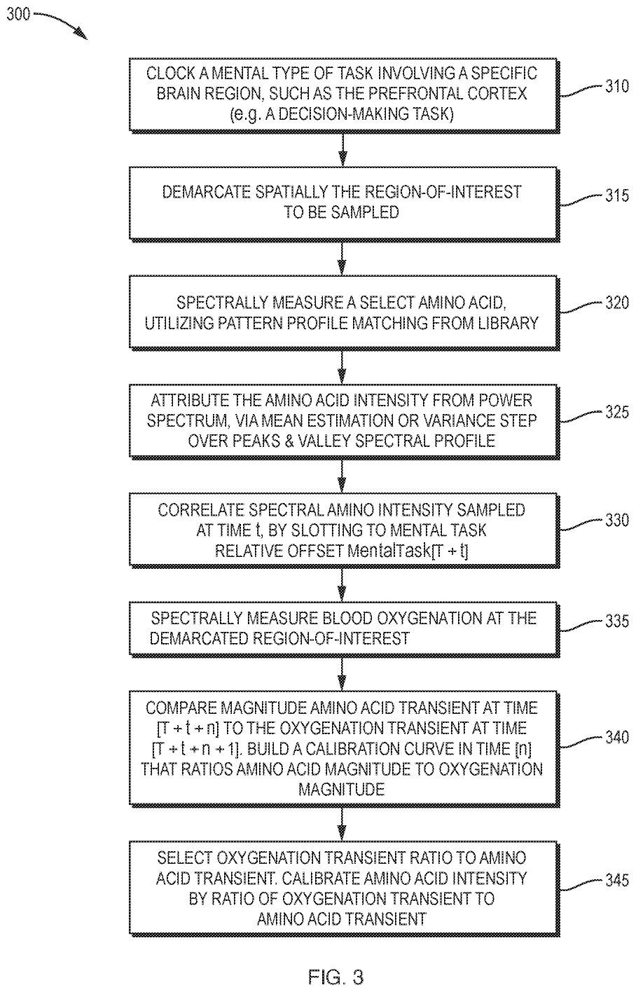

Device is placed on or close to forehead, at a location within the rectangle as shown in FIG. 1. FIGS. 2 and 3 supply flowchart steps for potential high-level execution involved in processing.

From RGB-based color space in camera imagery, the green channel is selected as optimal for cardiac activity (and with less motion artifacts than the red channel, higher penetration depth than the blue channel). Any areas of rhythmic pulsation, consonant with a potential pulsewave time period (normally 60-100 pulsewaves per minute) is identified. A confidence level through viewing target areas over a threshold time interval is established.

A region of interest surrounding each pulsation area is established. Because of multi-layer skin optical scattering, the region of interest must be chosen carefully to be as broad as practical given that neuronal tissue uptake must be very proximate to such vascular features. A region of interest is then shape-bounded in the image x, y pixel dimensions and sufficiently proximate to the pulsation area.

Given each region of interest, the surrounding pixelized area within bounds can be transformed into a shape-bounded-search-results image. As the device's imaging sensor can see from visible to near infrared spectrum, the resulting search-results sub-images can be transformed into Fourier domain. Utilizing spectral response for specific ions or molecules from a library of spectral profiles in the near infrared spectrum where tissue penetration is greater, a spectral filter can be created in the same [i, j] dimensions as the search-results image. For example, neurotransmitter or neuromodulators both play an important function in brain processing.

Spectral filter by the search-results image for each [i, j] pixel is multiplied and results in a NIR grayscale intensity image of the image in [i, j] dimensions.

At each grayscale pixel in such an image I[i, j], a postprocessing transform module can convert each pixel or grouped pixel intensity to an imputed value within relative physiological processing frequency range. For example, the darkest pixel or group might transform as a high frequency, e.g. 50 Hz or more. The lightest pixel or group might transform as a lower (waking state) frequency, e.g. 8 Hz. (Transforms can shift outcome value range for different tasks or individuals.)

Transformed [i, j] images now containing imputed pixel-level or pixel cluster-level frequency values are ordered in sampled time of procurement. At each pixel or cluster level, a frequency value is extracted in the first time interval. The same pixel or cluster level is extracted in the second time interval. Frequency value at time 1 is subtracted from frequency value at time 2. Subtraction shows the change in frequency value from time 1 to time 2, and hence represents change in measured frequency activity there. For example, if time 1 transformed image I [1, 1] pixel value is 40 Hz in frequency, and time 2's corresponding I [1, 1] pixel value is 50, then a change vector at first pairing saves as 10 (ChangeVector[1]=10).

Subtractive frequency values by successive image pairs across each [i, j] pixels or clusters are successively stored into an effective vector built from each image pair in image I1 [i, j] and image I2 [i, j]. The Change Vector1, once completed, has assessed all pixels or clusters across the two images.

Then the next image I3 [i, j] sequenced in time is obtained. The previous time 2 is reset as the next time 1. The new time 2 observations are obtained from this next image I3 [i, j]. The new time 1 observations are reset, to refer to the previous image I2 [i, j]. Time 2-Time 1 values are calculated again for each of the same pixel or cluster [i, j] as previously, and the new Change Vector2 is stored as the second column in a matrix.

Thus, a Change Series Matrix matrix of vector paired observations is built up. Change Series Matrix vectors are stored as successive columns by progressing through each successively time-based image to subtractive pairings stored as the next Change Vector in the matrix. This is to regularize the ensuing matrix dimensionality of same-length vector frequency changes to an [i, j] matrix for the next step.

Then the principal uncorrelated dimensions of the Change Series matrix need to be found. In a simple two vector matrix example (i.e. where two subtractive pairings were yielded from three image [i, j] observations sequenced in progressive time) such as the following:

| ChangeVector[1] | ChangeVector[2] |
|---|---|
| 70 | 30 |
| 60 | 20 |
| 60 | 30 |
| 40 | 40 |

The (variance) covariance matrix derives as:

| | |
|---|---|
| [118.8 (Var) | −50 (Cov) |
| −50 (Cov) | 50 (Var)] |

The negative values in the covariance matrix entries would indicate that there is negative correlation between Change Vector observations.

PCA involves diagonalizing the covariance matrix, to ensure all variables are independent. First the mean is subtracted from all elements. If the data is not scaled well to each other, dividing by the standard deviation may be needed. A linear combination of the covariance elements is calculated, such that each diagonal variable is uncorrelated. To determine the first principal component, the largest eigenvalue will correspond to the eigenvector expressing the largest variance in the data. To select the next principal component, the largest component can be subtracted out from the data to achieve a somewhat-flattened dataset. Then the process can be repeated until sufficient top components result for input to classifiers.

Each extracted frequency set produces its own particular set of PCA scores, so by examining the PCA factor space in preprocessing, evaluation can determine how much a target analyte's spectral features are represented in that factor space. Then the next extracted frequency set from the next subimage would be obtained. Each extracted frequency dataset can be rank-ordered by the strength of its PCA scores for the desired analyte, and the best one or a composite is used. (If insufficient target spectral features are found in any shape-bounded search space, then data extraction at target+neighboring frequencies (due to noise and scattering) may need to occur before performing PCA, as other analytes may have been relatively predominant in the original dataset.)

A Bayes classifier can take principal component input or the covariance matrix where such feature vectors are independent. It is a conditional probability model, where given covariance matrix or top PCA component vectors as input, probabilities via probability tables could be assigned to possible class outcome. From our example, the Covariance matrix:

| | |
|---|---|
| [118.8 (Var) | −50 (Cov) |
| −50 (Cov) | 50 (Var)] |

can be compared against a lookup table of prior library matrices, and when matched then obtain an associated class conditional probability from the Lookup table of priors. For example, perhaps the Lookup table contains a matching entry, and the corresponding conditional probability of healthy status given this individual's Covariance matrix yields 0.75. As it is found over a threshold margin designated for example as 0.5, this individual would classify as healthy. Methods of thresholding class probability to determine classification are known in the art. For example, Ehsani-Moghaddam et al, 2018 developed and applied a Naïve Bayes Classifier for diagnosis of Hunter syndrome. Luethy 2023 developed a genetic optimization algorithm for the determination of decision thresholds for multiple decision regions.

Lookup matching may also contain fuzzy matching, where an interval range might be used to match for the respective input values (as often occurs because individuals can neurally process a little differently from each other) if no exact match.

A naive Bayes classifier processes such on current component vectors or covariance matrix alone, but can be processing-intractable with large feature sets. Using prior classification as well, the likelihood of the same classification again can be expressed as the prior class probability multiplied by the conditional probability of the new feature vector falling into that class. (The denominator conditional probability of such feature vector does not need to be incorporated, as the numerator is equivalent to a joint probability model of a class and a feature vector together.) Such joint (or conditional in naive Bayes) probability results in a value between 0 and 1 (e.g. certainty), of such feature vector belonging to such class.

As indeed more than one top principal component may significantly boost the confidence level in outcome, top principal components can be concatenated as input to classification or the entire covariance matrix may be utilized. Classifiers are modifiable as to optimal input form and basis. For a covariance matrix or concatenated principal component vector, a conditional probability of belonging to a certain class result in a value between 0 and 1 (where I would then denote complete confidence). This output value then results in the individual falling somewhere given principal component dimensions, within a two-part boundary class space designated for example as healthy or conversely not (i.e. non-typical).

Class boundaries include some threshold margin where it might not be clear as to which class such individual belongs. If class probability of “healthy” for example is above a threshold margin, then the individual is designated as in the healthy class. If class probability of “healthy” is below a threshold margin, then the individual is designated as belonging in the other class, non-typical or unhealthy.

To calibrate the results, an EEG or NIR spectroscopic headset can be utilized. FIG. 3 illustrates how analyte concentration intensity might then be compared to accepted measurements such as EEG readings for the same individual. Alternatively, oxygenation vs deoxygenation hemoglobin concentration changes from variations in absorbed near infrared light with a NIR spectroscopic headset might be utilized for the same calibration purpose. Blood oxygenation may be measure by common techniques, such as blood-oxygen-level-dependent (BOLD) imaging.

For an analyte, both activity and concentration base need to be assessed, for optimal health.

To estimate a concentration base for an analyte, the power spectrum of a Fourier transform of an underlying analyte estimated signal is performed. Since this is done across all frequency range, the spectra represent the total concentration and after obtaining square root of each respective spike magnitude, they can be summed together.

Because these estimates may be off, it would be prudent to obtain other analyte's estimates and iterate towards fitting the curve given typical equilibrium ratios and performing ranges.

For a neuroprocessing-associated analyte's spectral components from a top candidate PCA factor space for which the variance is most numerically maximized, the power spectrum via Fourier transform is used. It is then helpful to identify any cyclic frequency synchronization viewed in Fourier frequency space. Thus, if the same magnitude spike repeats every 2*pi increment along the frequency axis, its occurrence as carrier phase shifts can be identified and then eliminated. The signal is therefore both more straightforward and an indicator of more intense neural processing.

In unpacking the encoding content from a carrier frequency, the cyclic spikes are first removed from such Fourier plot. Then magnitudes of successive spikes are each divided by the cyclic magnitude, as content encoding had been previously convolved (i.e. multiplied) by the carrier frequency. This represents sampled encoding content spanning the frequency domain.

The deconvolved frequency plot could be compared to others for classification purposes in the frequency domain, or converted back to time domain for such comparison.

In the frequency domain or in the time domain, evaluating the plot against classification of healthy or non-typical populations could be useful. For example, this could aid parents who question their child's struggles with schoolwork, or could aid students who wish to improve their performance.

In the frequency domain, evaluating the range of higher and lower frequencies could be useful. Higher frequencies might correspond to relative “edges” in processing, while lower presumably correspond relatively to content. With frequencies over or below associated high or low thresholds respectively, summing the frequency magnitudes to compare, averaging or utilizing each spike's magnitude via classifying could be progressive strategies. Issuing an alert or remote alerting to a parent/guardian if a subset of frequencies or if the average of such subset is above (or drops below) a threshold could be useful in say an inattention scenario. Alternatively, as high-frequency oscillations can sometimes, although not exclusively, mark a seizure, it could be useful to remote alert parents/guardians to uncover, or alert to an individual or staff to take precautions.

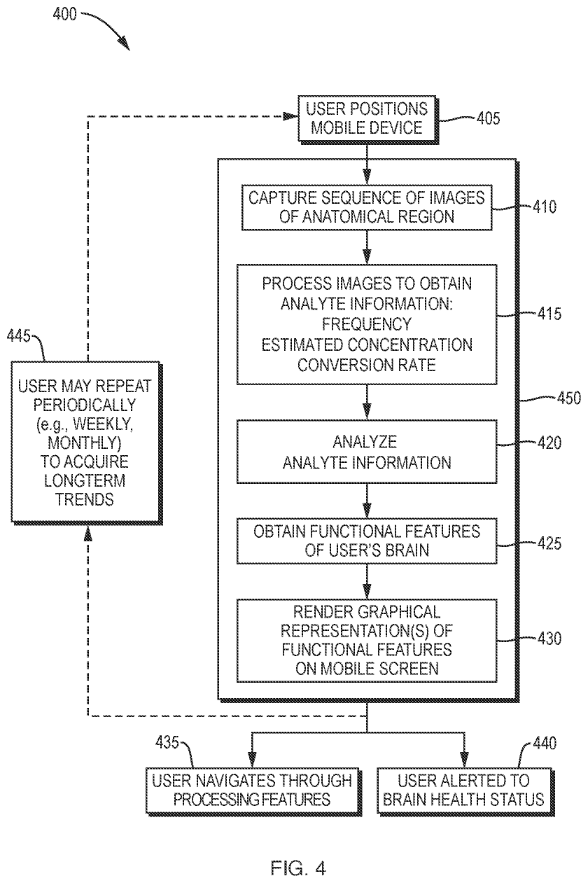

In the time domain, evaluating the converted plot as change in activity over time could be useful. The entirety or time domain subsets could also be useful to compare, via others. Classifying as healthy or non-typical against populations could help in assessment. Additionally, alerting if a sequence or subset target is reached, and/or evaluating how long it takes to reach a sequence or subset target, and comparing that to populations via classifying. For a defined sequence or wild-carded sequence, evaluating how long it takes to perform that sequence, and comparing it to other populations again as Healthy or Non-Typical. An embodiment 400, illustrated in FIG. 4, includes positioning a mobile device, having at least one camera equipped to capture photographs in the RGB and near-infrared spectrum in front of the face, head, and/or neck of the individual, e.g. the user. The mobile device captures a sequence of images of an anatomical region of the user, as shown in step 410. The analyte spectrographic data provides information relating to curve frequencies, concentration and conversion rate (step 415). This information is analyzed (step 420) to produce functional features of the user's brain (step 425), which is rendered graphically on a screen of the device for user viewing (step 430). Graphical rendering (step 430) can include and represent data in a three-dimensional manner. The user is then able to navigate through a mapping of the user's functional features while viewing real-time information relating to processing features (step 435). Additionally, the user can be alerted to brain health status, including any potentially negative markers of brain health (step 440). The user may repeatedly capture images to acquire long-term trends (step 445), in which case the mobile device captures additional data points and the user can be provided with additional and historical data relating to the user's brain health.

For someone struggling with mental processing, an additional alert type might be set as audio or for hard of hearing, in flashing large type or colors to encourage self-improvement.

Because it is hard to both hold a portable device to the forehead and look at an associated screen, it may be that sometimes a user would look at one screen and assess at forehead with another.

Assessing in multiple domains together may make sense sometimes. For example, if developing a longitudinal frequency assessment, where specific current time-based plotted steps need evaluating. For example, an emerging brain tumor may sometimes evidence in increased delta rhythm during wakefulness, relatively to prior evaluations.

Alternatively, by assessing wave directionality to see if slow waves are moving in opposite directions, which may indicate mild traumatic brain injury or post-traumatic stress disorder (PTSD). Also evaluating whether Fourier slow frequency spikes are increased in magnitude relative to previous assessments or to populations, which would lean towards possible mild traumatic brain injury. Next evaluating whether Fourier slow frequency spikes are decreased in magnitude relative to previous assessments or to populations, which would lean towards possible PTSD.

At the end in addition to appropriate points within the application's execution, associated brain function, data, and profile can be retained within the application's database.

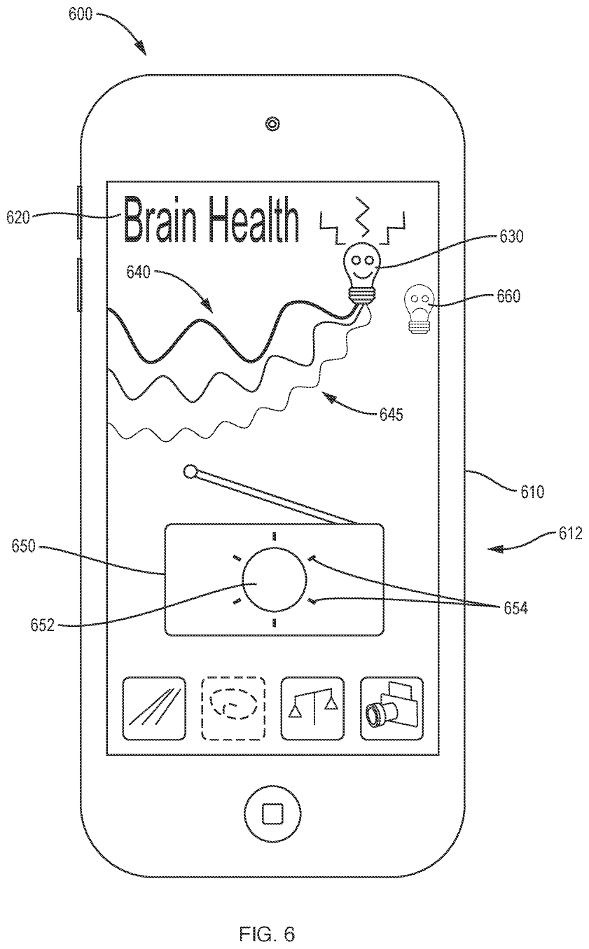

In one embodiment, the invention is directed to a mobile device for measuring and displaying brain health of an individual. The mobile device 500 includes display 510 which provides user interface 512 (including icons 530, 535, 540 and 545) is shown in FIG. 5A-5C. Near infrared sensor 502, processor 504 and camera 505 are included in the mobile device. Brain icon 535 is selected, therefore highlighted. Start display is of person's face 520 (FIG. 5A). Gradually, hair 555 fades to background or dissolves, increasingly replaced by the following screen. In FIG. 5B, Brain waves 565 circulate, sometimes rotating. Several carrier wave frequencies display in different amplitudes, phases, colors and/or line weights. Gradually, other face features 560 may substitute by simplifications (e.g. outlining) or graphics, or fade. If person is classified as ‘healthy’ in their brain by the application, a lit-up lightbulb 570 can appear and/or a smiley face, as shown in FIG. 5C. Lurking in the background can be the alternative for comparison purposes, such as a sad face and/or dimmed bulb 580. The person's head can be visually zoomed in yet also less prominent, as app results are displayed. If the person taps or clicks on the “healthy” lightbulb, a visual explanation appears on the next screen, as for example illustrated in FIG. 6.

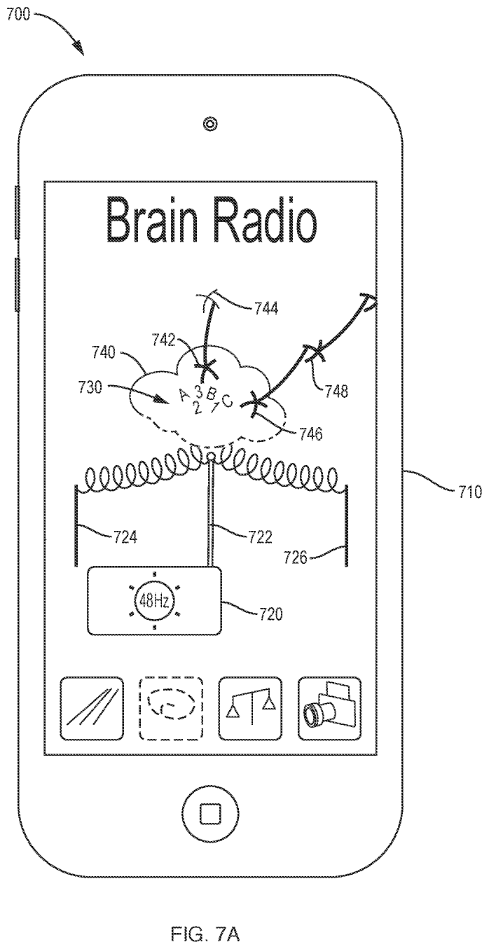

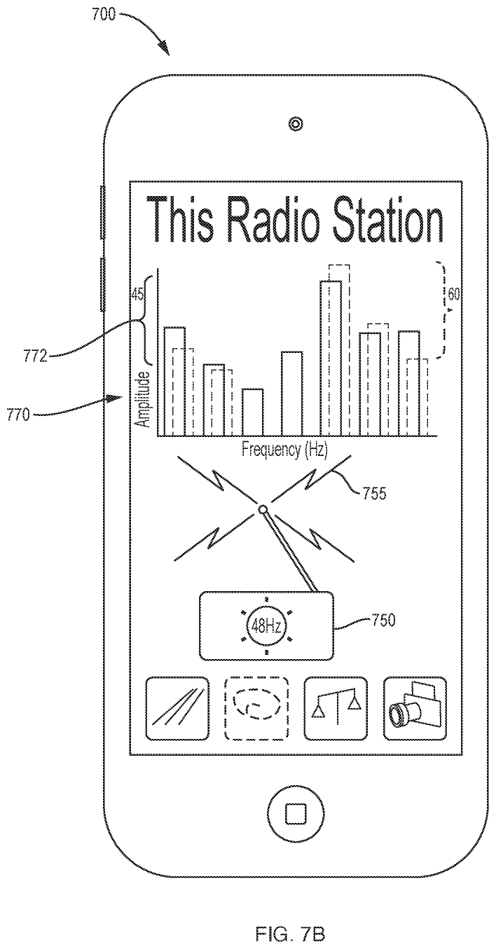

Mobile device 600 shows user interface 612 in FIG. 6. Brain health 620 as classified is displayed on screen 610. Separate split-content tiling also encourages user to peek at one's own mentally working “radio” 650. The “healthy” lightbulb 630 with emitting light gradient carries over from the prior screen. Weighted top components, of frequency contribution to healthy classification, are approximated and animated. In order to visually show weighted contributions to classifying, animated curves are line-weighted (e.g., 640, 645) and flow into the lightbulb. Curves spiral due to electrochemical B fields that are 3-dimensional rather than flat 2-D. There are other analogies that could visually explain electrical firing and encoding, but a radio 650 may be the most easily understood. This two-way radio has a tuning dial 652 (and eventually other controls). A circular dial will allow a better future display of time:frequency conversions. Perhaps six tick marks 654 span the dial, roughly for the five commonly-understood carrier frequency bands. If the user were to taps or click on a tick mark, curved-bar segment or perhaps even the dial, the next screen would invoke (FIG. 7A). The “not typical” dimmed or sad lightbulb 660 may still be lurking in the background for comparison purposes, under some conditions.

An embodiment 700, illustrated in FIG. 7, includes sample center and sideband frequencies, symbolic indication of what they might conceptually carry, and a demonstration of how that information would carry via sample neuronal interchange.

FIG. 7A illustrates device 700, including display 710 and user interface 712, which shows gamma-band 2-way radio “station” 720. The center frequency can be quantified in a radio dial in Hertz cycles per second, e.g., 48 Hz as shown. Spectral sidebands perhaps display, as a pair (narrowband) 724, 726 or more (wideband) (not shown). Light animation, which pulses up center 722 (e.g., center frequency) and sideband spikes, leads to pulsing spiral light animations towards a shaded jumble of (partial) letters and numbers 730 over a gradient cloud of light 740. As spike magnitude indicates intensity, a larger sideband may lead to relatively more spirals from that sideband. The gradient light cloud emanates outward, lighting up nearby neuronal dendritic branches and axon (742, 746) to signify information carries within and can propagate. In a weaker gradient, the next neuron 744 may not light up or propagate content. From a stronger gradient level, the next neuron 748 may light up and propagate accordingly. Information may bob, weave or dissolve (748) and reappear to denote encoding. More neurons may display in the foreground or background.

If the user touched sidebands, center frequency or the radio on previous screen, then a sample decoded readout of instantaneous frequencies in time displays (FIG. 7B). For the same visual task, a comparative readout of the general population also displays in dotted, shaded or different color form. To do so, the signal is preferably recentered around 0, and to determine the instantaneous frequency from rate of change in phase, a polar discriminator between successive samples (or samples since last influx or cardiac cycle start) of the complex valued signal can be used.

As one simple measure, the spread between lowest and highest consecutive frequencies per time in decoded sample can be displayed (772). The user can see how he compares to the greater group or population, as such could be seen as an indicator of encoding sensitivity. Samples may or may not be spaced out for viewing case, and they may be of different durations. Audio might accompany visual content.

Radio antenna might display relative power here, via line weight and length strength of emitting beams 755. This helps the user see that brain processing requires real energy to store and maintain (say against memory loss). Relative power is roughly proportional to the frequencies encoded.

FIG. 8 illustrates a computer network 110 or similar digital processing environment in which the present invention may be implemented. Mobile device or computer 50 and server computer(s) 60 provide processing, storage, and input/output devices executing application programs and the like. Mobile device or computer 50 can also be linked through communications network 70 to other computing devices, including other client devices/processes 50 and server computer(s) 60. Communications network 70 can be part of a remote access network, a global network (e.g., the Internet), a worldwide collection of computers, Local area or Wide area networks, and gateways that currently use respective protocols (TCP/IP, Bluetooth, etc.) to communicate with one another. Other electronic device/computer network architectures are suitable.

FIG. 9 is a diagram of the internal structure of a computer (e.g., mobile device/computer 50 or server computers 60). Mobile device or computer 50 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, microphone, displays, cameras, spectroscopy devices, wearable aids, speakers, etc.) to the computer 50, 60. Network interface 86 allows the computer to connect to various other devices attached to a network (e.g., network 70 of FIG. 8). Memory 90 provides volatile storage for computer software instructions 92 and data 94 used to implement an embodiment of the present invention (e.g., measurement module/submodules, comparator, identification module/submodules, probability engine/module including evaluator and display/reporter module, and supporting code detailed above). Disk storage 95 provides non-volatile storage for computer software instructions 92 and data 94 used to implement an embodiment of the present invention. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

### REFERENCES

D. J. Mehagnoul-Schipper et al Simultaneous Measurements of Cerebral Oxygenation Changes During Brain Activation by Near-Infrared Spectroscopy and Functional Magnetic Resonance Imaging in Healthy Young and Elderly Subjects, Human Brain Mapping, 2002.
Ehsani-Moghaddam B, Queenan J A, Mackenzie J, Birtwhistle, R V. Mucopolysaccharidosis type II detection by Naïve Bayes Classifier: An example of patient classification for a rare disease using electronic medical records from the Canadian Primary Care Sentinel Surveillance Network. PLOS One. Dec. 19, 2018. https://doi.org/10.1371/journal.pone.0209018.
Luethy R, Buturovic L. Decision Threshold Optimization for Diagnostic Tests using a Genetic Algorithm”, Inflammatrix, Published online Mar. 15, 2023.
Strangman et al., “Non-Invasive Neuroimaging Using Near-Infrared Light” Biol. Psychiatry 2002; 52:679-693.
White et al., “Laboratory spectra of CO₂ vibrational modes in planetary ice analogs” Icarus 2012; 221:1032-1042.
Huber et al., “Carbonic Acid Revisited: Vibrational Spectra, Energetics and the Possibility of Detecting an Elusive Molecule” AIP Advances 2012; 2:032180.
Biermann and Gilmour, “Hydronium Ion Absorption in the Region 1.7μ” Can. J. Chem. 1959, 37:1249-1253.
Saddawi-Konefka and Bryner, Chapter 1-Dissolved Oxygen in the Blood and Chapter 2-Bound Oxygen in the Blood, accessed on Oct. 23, 2014, http://www.umich.edu/˜projbnb/cvr/o2.html, 10 pages.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method for assessing brain frequencies in an individual, the method comprising:
a) positioning a near infrared spectroscopic device near an individual at an anatomical region to be studied;
b) determining, with the device at the anatomical region, a first frequency measurement of at least one molecule at a first time;
c) determining, with the device at the anatomical region, a second frequency measurement of the at least one molecule at a second time;
d) comparing the first frequency measurement to the second frequency measurement to generate a comparison;
e) identifying principal uncorrelated dimensions of the comparison to yield a covariance matrix;
f) utilizing Bayes or other classification on the covariance matrix to yield a resulting conditional class probability; and
g) thresholding the class probability to determine a classification for the individual's neural processing, the classification indicating a health status of the neural processing.

2. The method of claim 1, wherein the first time is a first time interval of less than 5 seconds, and the second time is a second time interval of less than 5 seconds.

3. The method of claim 1, further comprising converting the first and second frequency measurements to time-based signal samples.

4. The method of claim 1, further comprising converting the first and second frequency measurements to a sequence flowcharting sample.

5. The method of claim 1, further comprising, using time-based observations, identifying a maximum or minimum frequency from at least one of the first and second frequencies.

6. The method of claim 1, further comprising comparing an average of the first and second frequency measurements against a threshold value.

7. The method of claim 1, further comprising establishing a baseline from one or more signals of the at least one molecule at the first time, wherein the baseline represents a brain frequency pattern profile of the individual.

8. The method of claim 7, further comprising normalizing the brain frequency pattern profile based on at least one of age and gender of the individual.

9. The method of claim 1, wherein the at least one molecule includes an amino acid, the amino acid being tyrosine or phenylalanine.

10. The method of claim 1, wherein the at least one molecule includes an amino acid, the amino acid being a chemically-derivatized amino acid relating to dopamine.

11. The method of claim 10, wherein the chemically-derivatized amino acid is L-dihydroxyphenylalanine (L-DOPA) or dopamine.

12. The method of claim 1, further comprising measuring cerebral blood pressure, cerebral blood flow, cerebrospinal fluid pressure, cerebrospinal fluid flow, intracranial pressure, or combinations thereof.

13. The method of claim 1, wherein the region comprises a forehead of the individual.

14. The method of claim 1, wherein the region comprises a frontal, parietal, occipital, limbic, or temporal lobe of the individual.

15. The method of claim 1, wherein the first frequency measurement comprises a series of first frequency measurements and the second frequency measurement comprises a series of second frequency measurements, and wherein the comparing the first frequency measurement to the second frequency measurement comprises comparing the series of first frequency measurements to the series of second frequency measurements to generate the comparison as a comparison matrix.

16. A computer system to assess brain frequencies in an individual, the system comprising:
a) a measuring module configured to determine a first frequency measurement of at least one molecule at a first time; and configured to determine a second frequency measurement of the at least one molecule at a second time;
b) a comparison module configured to receive and compare the first measurement to the second measurement of the at least one molecule;
c) an identification module coupled to the comparison module and configured to identify principal uncorrelated dimensions of the comparison to yield a covariance matrix; and
d) a probability module coupled to the identification module and configured to utilizing Bayes or other classification on the covariance matrix to yield a resulting conditional class probability;
e) the probability module configured to threshold the class probability to determine a classification for the individual's neural processing, the classification indicating a health status of the neural processing.

17. The computer system of claim 16, wherein the first time is a first time interval of less than 5 seconds, and the second time is a second time interval of less than 5 seconds.

18. The computer system of claim 16, further comprising a conversion module responsive to the measuring module and configured to (i) convert the first and second frequency measurements to time-based signal samples, (ii) convert the first and second frequency measurements to a sequence flowcharting sample, or both (i) and (ii).

19. The computer system of claim 16, wherein the identification module is further configured to, using time-based observations, identify a maximum or minimum frequency from at least one of the first and second frequencies.

20. The computer system of claim 16, wherein at least one of the comparison module and the probability module is further configured to compare an average of the first and second frequency measurements against a threshold value.

21. The computer system of claim 16, further comprising a baseline module responsive to the measuring module and configured to establish a baseline from one or more signals of the at least one molecule at the first time, wherein the baseline represents a brain frequency pattern profile of the individual.

22. The computer system of claim 21, further comprising a normalization module configured to normalize the brain frequency pattern profile based on at least one of age and gender of the individual.

23. The computer system of claim 16, wherein the at least one molecule comprises an amino acid, the amino acid being tyrosine, phenylalanine, or a chemically-derivatized amino acid relating to dopamine.

24. The computer system of claim 16, further comprising a second measuring module configured to measure cerebral blood pressure, cerebral blood flow, cerebrospinal fluid pressure, cerebrospinal fluid flow, intracranial pressure, or combinations thereof.

25. The computer system of claim 16, further comprising a device module configured to connect one or more near infrared spectroscopic devices operatively coupled to the measuring module.

26. The computer system of claim 25, wherein the near infrared spectroscopic device is, or is incorporated in, a portable device, such as a cell phone, tablet, laptop computer, or wearable aid.

27. The computer system of claim 26, wherein the near infrared spectroscopic device comprises a camera and is configured to measure the at least one molecule.

28. The computer system of claim 16, wherein the first frequency measurement comprises a series of first frequency measurements and the second frequency measurement comprises a series of second frequency measurements, and wherein the comparison module is configured to compare the series of first frequency measurements to the series of second frequency measurements to generate the comparison as a comparison matrix.

29. A computer-implemented method of assessing brain health in an individual, the method comprising:
a) using a mobile device having a camera to capture a sequence of images of an anatomical region of an individual;
b) in a digital processor associated with the mobile device, processing the images to obtain at least one of frequency, estimated concentration, and conversion rate information relating to an analyte of the individual;
c) analyzing the at least one of frequency, estimated concentration, and conversion rate information to obtain one or more functional features of the individual's brain; and
d) on a screen of the mobile device, rendering a graphical representation of the functional features of the individual's brain.

30. The method of claim 29, wherein the camera captures images in the red-green-blue (RGB) spectrum.

31. The method of claim 29, wherein the camera captures images in the near-infrared (NIR) spectrum.

32. The method of claim 29, wherein the functional features obtained include at least one of a frequency of a phenylalanine, a tyrosine, and a dopamine.

33. The method of claim 29, wherein the functional features are computed for the individual's anatomy and compared to functional features of a group or population to assess brain health of the individual.

34. The method of claim 29, further comprising alerting the individual to brain health status.

35. The method of claim 29, wherein the mobile device is a cell phone, tablet, laptop computer, wearable aid, or stand-alone device.

36. A mobile device for measuring and displaying brain health of an individual, the device comprising:
a) a camera configured to capture a sequence of images of an anatomical region of an individual;
b) a digital processor configured to process the images to obtain at least one of frequency, estimated concentration, and conversion rate information relating to an analyte of the individual;
c) the digital processor configured to analyze the at least one of frequency, estimated concentration, and conversion rate information to obtain one or more functional features of the individual's brain; and
d) a screen configured to render a graphical representation of the functional features of the individual's brain.

37. The device of claim 36, wherein the camera is configured to capture images in the red-green-blue (RGB) spectrum.

38. The device of claim 36, wherein the camera is configured to capture images in the near-infrared (NIR) spectrum.

39. The device of claim 36, wherein the functional features are computed for the individual's anatomy and compared to functional features of a group or population to assess brain health of the individual.

40. The device of claim 36, further comprising a module configured to alert the individual of brain health, a circulatory deficiency, or combinations thereof.
